(19) United States
(12) Patent Application Publication
(10) Pub. No.: US 2018/0117276 A1
(43) Pub. Date: May 3, 2018

(54) CHILDRENS GUIDED BREATHING AND MEDIATION, MINDFULNESS, AND YOGA TOY

(71) Applicants: Jesal Kantawala, Anaheim, CA (US); Anchal Chand, Chatsworth, CA (US)

(72) Inventors: Jesal Kantawala, Anaheim, CA (US); Anchal Chand, Chatsworth, CA (US)

(21) Appl. No.: 15/803,020

(22) Filed: Nov. 3, 2017

Related U.S. Application Data
(60) Provisional application No. 62/416,804, filed on Nov. 3, 2016.

(51) Int. Cl. A61M 21/02; A63H 33/22; A63H 5/00

(52) U.S. Cl. CPC A61M 21/02; A61M 2021/0044; A63H 5/00; A63H 33/22

(57) ABSTRACT

The present invention provides a children's guided breathing and meditation, mindfulness, and yoga toy. The children's toy includes a body containing one or more lights visible behind a translucent visor; a sound device that includes a flash memory integrated circuit; a microcontroller; an amplifier; a battery, and a speaker. The microcontroller is programmed with nature sounds recordings, guided breathing and meditation recordings, guided mindfulness recordings, and guided yoga recordings that when played by the child, are coordinated with light generated by the light emitting diodes (LEDs).

### RELATED APPLICATIONS

This application claims priority to U.S. Provisional Patent Application Ser. No. 62/416,804 filed Nov. 3, 2016, which is hereby incorporated by reference in its entirety for all purposes.

### BACKGROUND OF THE INVENTION

In modern society, children exhibit increased levels of stress, anxiety, and decreased wellness compared to previous generations. What is needed is an interactive children's toy that provides guided breathing and meditation, mindfulness, and yoga commands using a toy that the child can relate to and identify with.

### SUMMARY OF THE INVENTION

The present invention provides a children's guided breathing and meditation, mindfulness, and yoga device. The children's device includes a body containing one or more lights visible behind a translucent visor; a sound device that includes a flash memory integrated circuit; a microcontroller; an amplifier; a battery, and a speaker. The microcontroller is programmed with nature sounds recordings, guided breathing and meditation recordings, guided mindfulness recordings, and guided yoga recordings that when played by the child, are coordinated with light generated by the light emitting diodes (LEDs).

The present invention provides a children's guided breathing and meditation, mindfulness, and yoga toy that is universal serial bus (USB) compatible, allowing more recordings of meditation and breathing sounds to be uploaded from the computer or external device. The guided breathing and meditation, mindfulness, and yoga instructions are recorded with child voice or character voice for commonality and familiarity with children. The child voice or character voice teaches mindfulness and yoga with children for familiarity and active fun. The toy is portable so that it can be used in classrooms and other settings in a figure that the children find reassuring and lovable. Further, the guided breathing and meditation, mindfulness, and yoga may be updated using the universal serial bus (USB) port once they have been mastered and can be custom tailored to the age of the child.

The present invention provides a children's guided breathing and meditation, mindfulness, and yoga toy. The children's toy includes: a body having a first surface, a second surface, a cavity, and a visor opening, wherein the body includes; one or more light sources each independently positioned within the cavity; and a translucent visor covering the visor opening; a sound device positioned within the cavity; wherein the sound device includes: a flash memory integrated circuit; a microcontroller; an amplifier; and a power source, wherein the microcontroller is in electrical communication with one or more buttons on the first surface of the body, the amplifier, and a power source, wherein the microcontroller is programmed to have one or more nature sounds recordings, one or more guided breathing and meditation recordings, one or more guided mindfulness recordings, and one or more guided yoga recordings, wherein the amplifier is in electrical communication with a speaker on the first surface of the body and the flash memory integrated circuit; and wherein the one or more light sources are in electrical communication with the microcontroller.

In one embodiment, the one or more light sources each independently include one or more light emitting diodes (LEDs). In one embodiment, the one or more nature sounds recordings, the one or more guided breathing and meditation recordings, the one or more guided mindfulness recordings, and the one or more guided yoga recordings are each independently coordinated with light generated by the one or more light sources. In one embodiment, the one or more buttons are used to activate the sound device to broadcast one or more nature sounds recordings through the speaker and to activate the one or more light sources to emit one or more colors. In one embodiment, the one or more nature sounds recordings, the one or more guided breathing and meditation recordings, the one or more guided mindfulness recordings, and the one or more guided yoga recordings are each independently coordinated with one or more light effects generated by the one or more light sources.

In one embodiment, the one or more light effects cause the one or more light sources to fade in and to fade out. In one embodiment, the body include one or more metals, one or more plastics, one or more wooden materials, or a combination thereof. In one embodiment, the body include one or more plastic materials. In one embodiment, the body include one or more rigid plastic materials.

In one embodiment, the one or more nature sounds recordings, the one or more guided breathing and meditation recordings, the one or more guided mindfulness recordings, and the one or more guided yoga recordings are each independently provided using a child's voice that the child can relate to.

In one embodiment, the children's guided breathing and meditation, mindfulness, and yoga toy further includes an on/off power switch. In one embodiment, the on/off power switch is a push switch. In one embodiment, the children's guided breathing and meditation, mindfulness, and yoga toy further includes including a volume switch. In one embodiment, the power source is a battery. In one embodiment, the battery is rechargeable. In one embodiment, the guided breathing and meditation, mindfulness, and yoga toy includes a sleep mode that turns off when the child is going to sleep.

In one embodiment, the one or more nature sounds recordings each independently include one or more ocean sounds, one or more forest sounds, one or more space sounds, one or more animal sounds, one or more wind sounds, or a combination thereof.

In one embodiment, the one or more guided breathing and meditation recordings each independently include guided inhales and exhales or a combination thereof. In one embodiment, the one or more guided mindfulness recordings each independently include children's voice character of guided meditations through voice with musical accompaniment; including instructions to close eyes, relax body, breathing in and out, or a combination thereof. In one embodiment, the one or more guided yoga recordings each independently include voice of character teaching yoga stretches and postures or a combination thereof.

In one embodiment, the children's guided breathing and meditation, mindfulness, and yoga toy further includes one or more universal serial bus (USB) ports.

The present invention provides a children's guided breathing and meditation, mindfulness, and yoga toy. The children's toy includes: a sitting yogi having a first surface, a second surface, a cavity, and a visor opening, wherein the sitting yogi includes; a first sound push button on the first surface of the sitting yogi; an on/off push button on the first surface of the sitting yogi; a second sound push button on the first surface of the sitting yogi; one or more light sources each independently positioned within the cavity; and a translucent visor covering the visor opening; a sound device positioned within the cavity of the sitting yogi; wherein the sound device includes: a flash memory integrated circuit; a microcontroller; an amplifier; and a power source, wherein the microcontroller is in electrical communication with the first sound push button, the on/off push button, the second sound push button, the amplifier, and a power source, wherein the microcontroller is programmed to have one or more nature sounds recordings, one or more guided breathing and meditation recordings, one or more guided mindfulness recordings, and one or more guided yoga recordings provided using a child's voice, wherein the amplifier is in electrical communication with a speaker positioned within the cavity of the sitting yogi and the flash memory integrated circuit; and wherein the one or more light sources are in electrical communication with the microcontroller.

In one embodiment, the one or more light sources include one or more light emitting diodes (LEDs). In one embodiment, the one or more nature sounds recordings, the one or more guided breathing and meditation recordings, the one or more guided mindfulness recordings, and the one or more guided yoga recordings are each independently coordinated with light generated by the one or more light sources. In one embodiment, the one or more buttons are used to activate the sound device to broadcast one or more nature sounds recordings through the speaker and to activate the one or more light sources to emit one or more colors. In one embodiment, the one or more nature sounds recordings, the one or more guided breathing and meditation recordings, the one or more guided mindfulness recordings, and the one or more guided yoga recordings are each independently coordinated with one or more light effects generated by the one or more light sources.

In one embodiment, the one or more light effects cause the one or more light sources to fade in and to fade out. In one embodiment, the sitting yogi includes one or more metals, one or more plastics, one or more wooden materials, or a combination thereof. In one embodiment, the sitting yogi includes one or more plastic materials. In one embodiment, the sitting yogi includes one or more rigid plastic materials.

In one embodiment, the children's guided breathing and meditation, mindfulness, and yoga toy further includes an on/off power switch. In one embodiment, the on/off power switch is a push switch. In one embodiment, the children's guided breathing and meditation, mindfulness, and yoga toy further includes a volume switch. In one embodiment, the power source is a battery. In one embodiment, the battery is rechargeable.

The present invention provides a children's guided breathing and meditation, mindfulness, and yoga toy. The children's toy includes: a sitting yogi having a first surface, a second surface, a cavity, and a visor opening, wherein the sitting yogi includes; a first sound push button on the first surface of the sitting yogi; an on/off push button on the first surface of the sitting yogi; a second sound push button on the first surface of the sitting yogi; one or more light emitting diodes (LEDs) each independently positioned within the cavity; and a translucent visor covering the visor opening; a sound device positioned within the cavity of the sitting yogi; wherein the sound device includes: a flash memory integrated circuit; a microcontroller; an amplifier; and a battery, wherein the microcontroller is in electrical communication with the first sound push button, the on/off push button, the second sound push button, the amplifier, and a battery, wherein the microcontroller is programmed to have one or more nature sounds recordings, one or more guided breathing and meditation recordings, one or more guided mindfulness recordings, and one or more guided yoga recordings provided using a child's voice, wherein the amplifier is in electrical communication with a speaker positioned within the cavity of the sitting yogi and the flash memory integrated circuit; wherein the one or more light emitting diodes (LEDs) are in electrical communication with the microcontroller, and wherein the one or more nature sounds recordings, the one or more guided breathing and meditation recordings, the one or more guided mindfulness recordings, and the one or more guided yoga recordings are each independently coordinated with light generated by the one or more light emitting diodes (LEDs).

In one embodiment, the children's guided breathing and meditation, mindfulness, and yoga toy further includes one or more universal serial bus (USB) ports.

The present invention provides a children's guided breathing and meditation, mindfulness, and yoga toy. The children's toy includes: an animal head having a first surface, a second surface, a cavity, and a visor opening, wherein the animal head includes; one or more buttons on the first surface of the animal head; one or more light sources each independently positioned within the cavity; and a translucent visor covering the visor opening; an animal body coupled to the animal head and having a first surface, a second surface, and a cavity, wherein the animal body includes: a sound device positioned within the cavity of the animal body; wherein the sound device includes: a flash memory integrated circuit; a microcontroller; an amplifier; and a power source, wherein the microcontroller is in electrical communication with the one or more buttons, the amplifier, and a power source, wherein the microcontroller is programmed to have one or more nature sounds recordings, one or more guided breathing and meditation recordings, one or more guided mindfulness recordings, and one or more guided yoga recordings, wherein the amplifier is in electrical communication with a speaker on the first surface of the animal body and the flash memory integrated circuit; and wherein the one or more light sources are in electrical communication with the microcontroller.

In one embodiment, the one or more light sources each independently include one or more light emitting diodes (LEDs). In one embodiment, the one or more nature sounds recordings, the one or more guided breathing and meditation recordings, the one or more guided mindfulness recordings, and the one or more guided yoga recordings are each independently coordinated with light generated by the one or more light sources. In one embodiment, the one or more buttons are used to activate the sound device to broadcast one or more nature sounds recordings through the speaker and to activate the one or more light sources to emit one or more colors. In one embodiment, the one or more nature sounds recordings, the one or more guided breathing and meditation recordings, the one or more guided mindfulness recordings, and the one or more guided yoga recordings are each independently coordinated with one or more light effects generated by the one or more light sources. In one embodiment, the one or more light effects cause the one or more light sources to fade in and to fade out.

In one embodiment, the animal head and the animal body each independently include one or more metals, one or more plastics, one or more wooden materials, or a combination thereof. In one embodiment, the animal head and the animal body each independently include one or more plastic materials. In one embodiment, the animal head and the animal body each independently include one or more rigid plastic materials.

In one embodiment, the children's guided breathing and meditation, mindfulness, and yoga toy further includes an on/off power switch. In one embodiment, the on/off power switch is a push switch. In one embodiment, the children's guided breathing and meditation, mindfulness, and yoga toy further includes including a volume switch. In one embodiment, the power source is a battery. In one embodiment, the battery is rechargeable.

In one embodiment, the children's guided breathing and meditation, mindfulness, and yoga toy further includes one or more universal serial bus (USB) ports.

The present invention provides a children's guided breathing and meditation, mindfulness, and yoga toy. The children's toy includes: a yogi head having a first surface, a second surface, a cavity, and a visor opening, wherein the yogi head includes; a first sound push button on the first surface of the yogi head; an on/off push button on the first surface of the yogi head; a second sound push button on the first surface of the yogi head; one or more light sources each independently positioned within the cavity; and a translucent visor covering the visor opening; a sitting yogi body coupled to the yogi head and having a first surface, a second surface, and a cavity, wherein the sitting yogi body includes: a sound device positioned within the cavity of the sitting yogi body; wherein the sound device includes: a flash memory integrated circuit; a microcontroller; an amplifier; and a power source, wherein the microcontroller is in electrical communication with the first sound push button, the on/off push button, the second sound push button, the amplifier, and a power source, wherein the microcontroller is programmed to have one or more nature sounds recordings, one or more guided breathing and meditation recordings, one or more guided mindfulness recordings, and one or more guided yoga recordings, wherein the amplifier is in electrical communication with a speaker positioned within the cavity of the sitting yogi body and the flash memory integrated circuit; and wherein the one or more light sources are in electrical communication with the microcontroller.

The present invention provides a children's guided breathing and meditation, mindfulness, and yoga toy. The children's toy includes: a yogi head having a first surface, a second surface, a cavity, and a visor opening, wherein the yogi head includes; a first sound push button on the first surface of the yogi head; an on/off push button on the first surface of the yogi head; a second sound push button on the first surface of the yogi head; one or more light emitting diodes (LEDs) each independently positioned within the cavity; and a translucent visor covering the visor opening; a sitting yogi body coupled to the yogi head and having a first surface, a second surface, and a cavity, wherein the sitting yogi body includes: a sound device positioned within the cavity of the sitting yogi body; wherein the sound device includes: a flash memory integrated circuit; a microcontroller; an amplifier; and a battery, wherein the microcontroller is in electrical communication with the first sound push button, the on/off push button, the second sound push button, the amplifier, and a battery, wherein the microcontroller is programmed to have one or more nature sounds recordings, one or more guided breathing and meditation recordings, one or more guided mindfulness recordings, and one or more guided yoga recordings, wherein the amplifier is in electrical communication with a speaker positioned within the cavity of the sitting yogi body and the flash memory integrated circuit; and wherein the one or more light emitting diodes (LEDs) are in electrical communication with the microcontroller.

The present invention provides a children's guided breathing and meditation, mindfulness, and yoga toy. The children's toy includes: a yogi head having a first surface, a second surface, a cavity, and a visor opening, wherein the yogi head includes; a first sound push button on the first surface of the yogi head; an on/off push button on the first surface of the yogi head; a second sound push button on the first surface of the yogi head; one or more light emitting diodes (LEDs) each independently positioned within the cavity; and a translucent visor covering the visor opening; a sitting yogi body coupled to the yogi head and having a first surface, a second surface, and a cavity, wherein the sitting yogi body includes: a sound device positioned within the cavity of the sitting yogi body; wherein the sound device includes: a flash memory integrated circuit; a microcontroller; an amplifier; and a battery, wherein the microcontroller is in electrical communication with the first sound push button, the on/off push button, the second sound push button, the amplifier, and a battery, wherein the microcontroller is programmed to have one or more nature sounds recordings, one or more guided breathing and meditation recordings, one or more guided mindfulness recordings, and one or more guided yoga recordings, wherein the amplifier is in electrical communication with a speaker positioned within the cavity of the sitting yogi body and the flash memory integrated circuit; wherein the one or more light emitting diodes (LEDs) are in electrical communication with the microcontroller, and wherein the one or more nature sounds recordings, the one or more guided breathing and meditation recordings, the one or more guided mindfulness recordings, and the one or more guided yoga recordings are each independently coordinated with light generated by the one or more light emitting diodes (LEDs).

In one embodiment, the children's guided breathing and meditation, mindfulness, and yoga toy further includes one or more universal serial bus (USB) ports.

### BRIEF DESCRIPTION OF THE DRAWINGS

Embodiments of the invention may be best understood by referring to the following description and accompanying drawings, which illustrate such embodiments. In the drawings:

FIGS. 1-5 are drawings of the various external views of the exemplary children's guided breathing and meditation, mindfulness, and yoga toy.

FIG. 6 is a wiring diagram of the interior components of the exemplary children's guided breathing and meditation, mindfulness, and yoga toy.

FIG. 7 is a drawing of the interior components of the exemplary children's guided breathing and mediation, mindfulness, and yoga toy

FIG. 8 is a block diagram illustrating an exemplary method of using the exemplary children's guided breathing and meditation, mindfulness, and yoga toy.

The drawings are not necessarily to scale. Like numbers used in the figures refer to like components, steps, and the like. However, it will be understood that the use of a number to refer to a component in a given figure is not intended to limit the component in another figure labeled with the same number.

### DETAILED DESCRIPTION OF THE INVENTION

The present invention provides a children's guided breathing and meditation, mindfulness, and yoga device. The children's device includes a body containing one or more lights visible behind a translucent visor; a sound device that includes a flash memory integrated circuit; a microcontroller; an amplifier; a battery, and a speaker. The microcontroller is programmed with nature sounds recordings, guided breathing and meditation recordings, guided mindfulness recordings, and guided yoga recordings that when played by the child, are coordinated with light generated by the light emitting diodes (LEDs).

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, M A, 1993 and The American Heritage Dictionary of the English Language, Houghton Mifflin, Boston Mass., 1981.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example, about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “comprising” or “comprises” is intended to mean that the compositions and methods include the recited elements, but not excluding others.

As used herein, the term “coupled” means the joining of two members directly or indirectly to one another. Such joining may be stationary in nature or movable in nature and/or such joining may allow for the flow of fluids, electricity, electrical signals, or other types of signals or communication between two members. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature.

As used herein, the phrase “operatively coupled” refers to bringing two or more items together or into relationship with each other such that they may operate together or allow transfer of information between the two or more items.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “proximal” refers to the closest end of an object. In contrast, the term “distal” refers to the farthest end of an object.

As used herein, the terms “front,” “back,” “rear,” “upper,” “lower,” “right,” and “left” in this description are merely used to identify the various elements as they are oriented in the FIGS., with “front,” “back,” and “rear” being relative apparatus. These terms are not meant to limit the element which they describe, as the various elements may be oriented differently in various applications.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

The invention illustratively disclosed herein suitably may be practiced in the absence of any element which is not specifically disclosed herein.

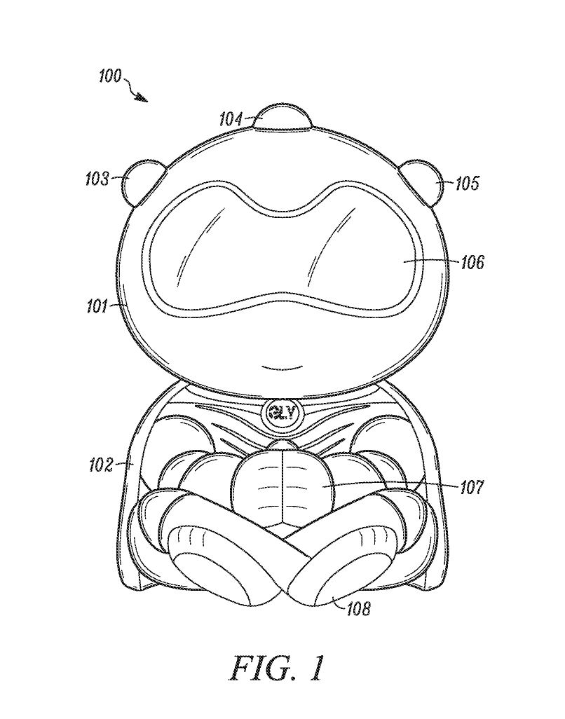

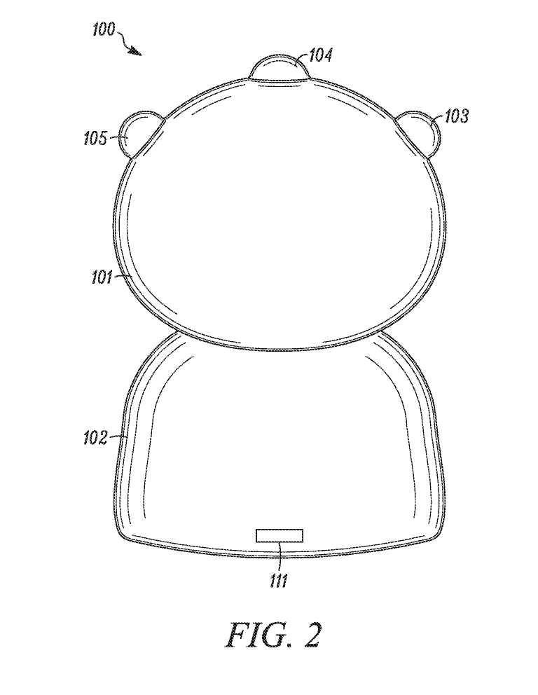

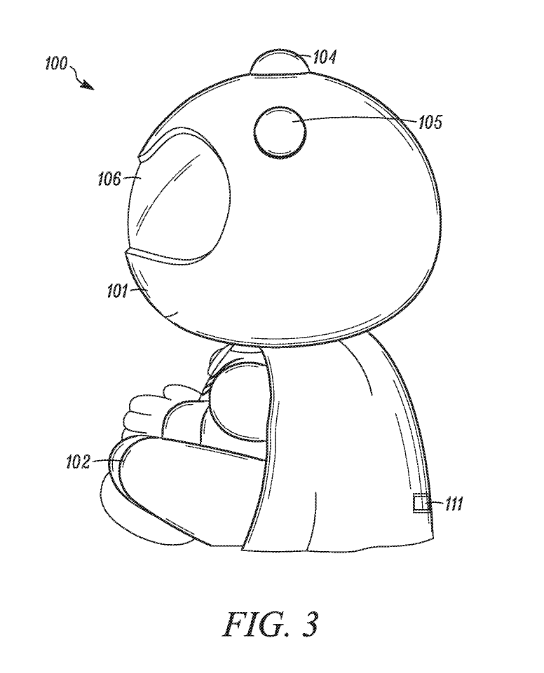

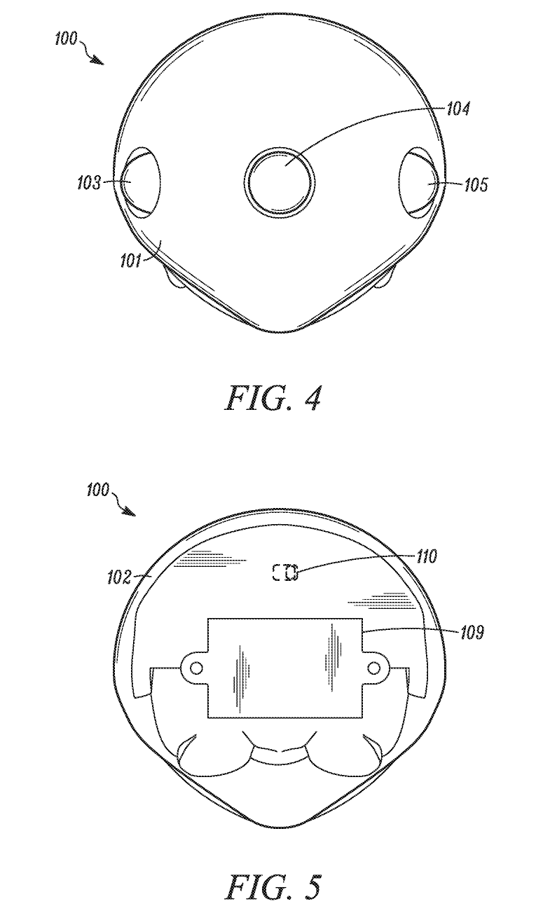

FIGS. 1-5 are drawings of the various external views of the exemplary children's guided breathing and meditation, mindfulness, and yoga toy 100. The children's guided breathing and meditation, mindfulness, and yoga toy 100 includes: a yogi head 101 and a sitting yogi body 102. The yogi head 101 includes a first sound push button 103, an on/off push button 104, a second sound push button 105, one or more light sources (not shown) positioned within the cavity of the yogi head 101; and a translucent visor 106 covering the visor opening (not shown). The sitting yogi body 102 includes a sound device (not shown), hands 107, feet 108, battery cover 109, power switch 110, and universal serial bus (USB) port 111. The universal serial bus (USB) port 111 may receive a universal serial bus (USB) memory device (not shown) that includes one or more uploaded routines, such as one or more nature sounds recordings, one or more guided breathing and meditation recordings, one or more guided mindfulness recordings, one or more guided yoga recordings, or a combination thereof.

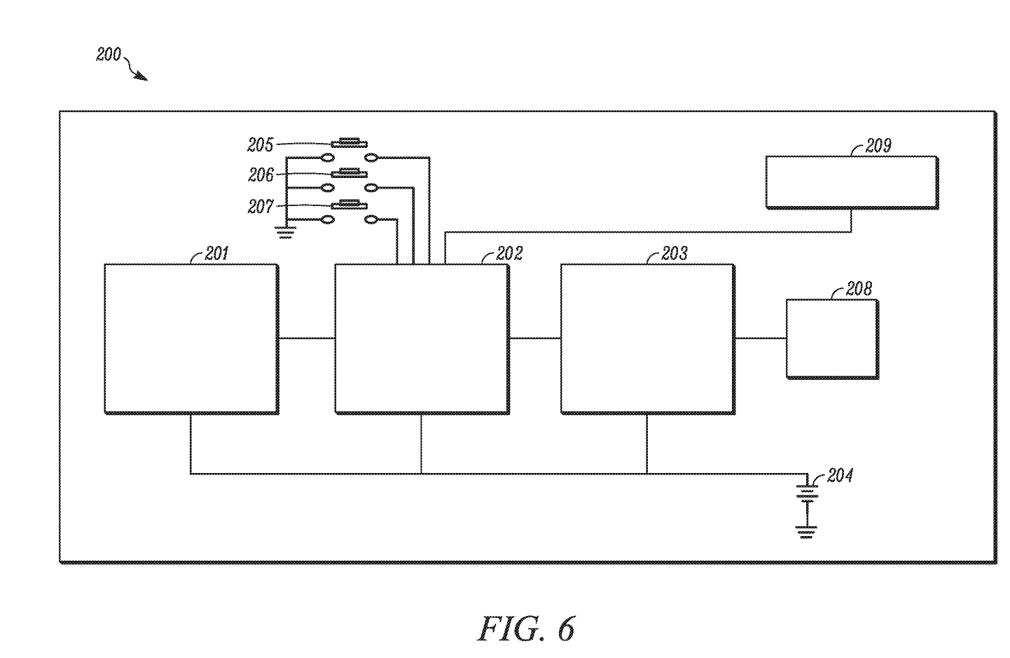

FIG. 6 is a wiring diagram of the interior components of the exemplary children's guided breathing and meditation, mindfulness, and yoga toy 200. The children's guided breathing and meditation, mindfulness, and yoga toy 200 includes: a sound device 201 includes: a flash memory integrated circuit 201; a microcontroller 202; an amplifier 203; and a power source 204. The microcontroller 202 is in electrical communication with the first sound push button 205, the on/off push button 206, the second sound push button 207, the amplifier 203, and the power source 204. The microcontroller 202 is programmed to have one or more nature sounds recordings, one or more guided breathing and meditation recordings, one or more guided mindfulness recordings, and one or more guided yoga recordings. The amplifier 203 is in electrical communication with a speaker 208 positioned within the cavity of the sitting yogi body (not shown) and the flash memory integrated circuit 201. The two light sources 209 are in electrical communication with the microcontroller 202.

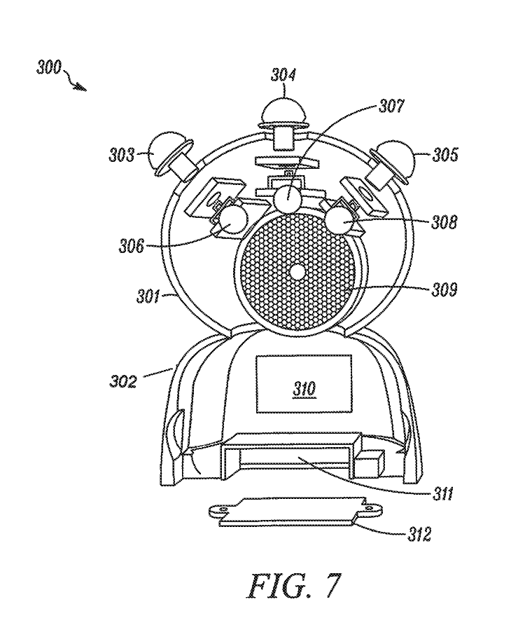

FIG. 7 is a drawing of the interior components of the exemplary children's guided breathing and mediation, mindfulness, and yoga toy 300. The children's guided breathing and mediation, mindfulness, and yoga toy 300 includes: a yogi head 301 and a sitting yogi body 302. The yogi head 301 includes a first sound push button 303, an on/off push button 304, a second sound push button 305, three light sources 306, 307, and 308; and a sound device 309. The yogi body includes a central processing unit 310, a power source 311, and a cover 312. In one embodiment, the three light sources 306, 307, and 308 are each light emitting diodes (LEDs).

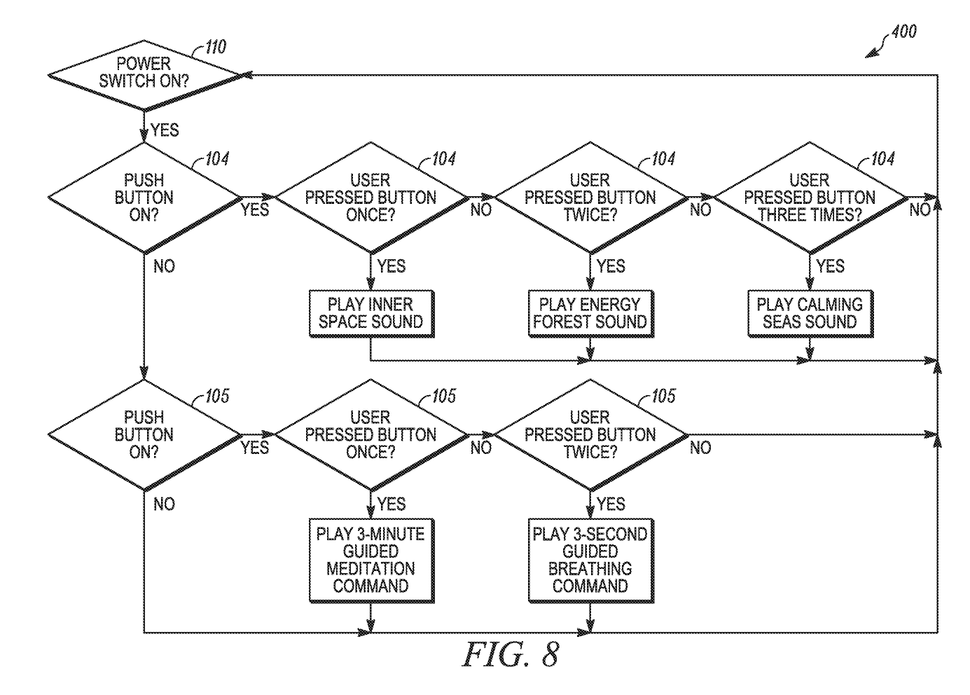

Operation of the exemplary children's guided breathing and meditation, mindfulness, and yoga toy of FIGS. 1-7 will now be explained with reference to FIG. 8, which shows the logic scheme 400 performed by the microcontroller. As indicated by FIG. 8, a child must turn on the electronics modules via the power switch 110 of FIG. 5 to provide sound and lighting effects in response to the child's interaction with the children's guided breathing and meditation, mindfulness, and yoga toy 100. The child pushes the on/off push button 104 to begin the session and activate the microcontroller 202.

The microcontroller 202 checks to see if the first sound push button 103 is depressed. If the first sound push button 103 is depressed, then the microcontroller 202 sends a three-minute nature sound to the speaker to be heard by the child and a signal to the two light sources to provide a specific light effect to be seen by the child through the translucent visor 106. The child can play various three-minute nature sounds (e.g., Inner Space, Energy Forest, Calming Seas, etc.) in a continuous fashion or skip to the next three-minute nature sound by depressing the first sound push button 103 as many times as desired. Each three-minute nature sounds (e.g., Inner Space, Energy Forest, Calming Seas, etc.) is linked to a particular color observed through the translucent visor 106, for example, purple, green, and blue, respectively.

If the microcontroller 202 determines that the first sound push button 103 is not depressed, the microcontroller 202 will check to see if the second sound push button 105 is depressed. If the second sound push button 105 is depressed, the microcontroller 202 sends a three-minute guided meditation voice teaching the child to breathe in and out, with positive affirmations. The microcontroller 202 also activates particular colors, which are observed through the translucent visor 106, for example, purple, green, and blue. If the second sound push button 105 is pressed again, the microcontroller 202 sends a four-second inhale and exhale command that lasts for 2 minutes and is accompanied with white light synchronized with the breathing.

As described above, the exemplary children's guided breathing and meditation, mindfulness, and yoga toy of FIGS. 1-7 use three buttons for operation, a first push button, an on/off push button, and a second push button which allow the user to cycle through the various one or more nature sounds recordings, one or more guided breathing and meditation recordings, one or more guided mindfulness recordings, and one or more guided yoga recordings. However, other embodiments utilize one button and two buttons.

In a one button embodiment, the user depresses the button to turn on the toy and to activate the one or more light sources. The user presses the button a second time to activate the deep breathing routine with the one or more light sources synchronized with them. When the user presses the button a third time, the toy will cycle through any uploaded track stored on a universal serial bus (USB) memory drive. When the user presses the button a fourth time, the toy will shut off.

In a one button embodiment, the user depresses the first button to turn on the toy and to activate the one or more light sources. The user presses the second button to activate the guided meditation routine. When the user presses the second button again, the toy will cycle through any uploaded track stored on a universal serial bus (USB) memory drive. When the user presses the first button again, the toy will shut off.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance or component fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention.

Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

What is claimed is:

1. A children's guided breathing and meditation, mindfulness, and yoga toy comprising:
a body having a first surface, a second surface, a cavity, and a visor opening, wherein the body comprises;
one or more light sources each independently positioned within the cavity; and
a translucent visor covering the visor opening;
a sound device positioned within the cavity;
wherein the sound device comprises:
a flash memory integrated circuit;
a microcontroller;
an amplifier; and
a power source,
wherein the microcontroller is in electrical communication with one or more buttons on the first surface of the body, the amplifier, and a power source,
wherein the microcontroller is programmed to have one or more nature sounds recordings, one or more guided breathing and meditation recordings, one or more guided mindfulness recordings, and one or more guided yoga recordings,
wherein the amplifier is in electrical communication with a speaker on the first surface of the body and the flash memory integrated circuit; and
wherein the one or more light sources are in electrical communication with the microcontroller.

2. The children's guided breathing and meditation, mindfulness, and yoga toy of claim 1, wherein the one or more nature sounds recordings, the one or more guided breathing and meditation recordings, the one or more guided mindfulness recordings, and the one or more guided yoga recordings are each independently coordinated with light generated by the one or more light sources.

3. The children's guided breathing and meditation, mindfulness, and yoga toy of claim 1, wherein the one or more buttons are used to activate the sound device to broadcast one or more nature sounds recordings through the speaker and to activate the one or more light sources to emit one or more colors.

4. The children's guided breathing and meditation, mindfulness, and yoga toy of claim 1, wherein the one or more nature sounds recordings, the one or more guided breathing and meditation recordings, the one or more guided mindfulness recordings, and the one or more guided yoga recordings are each independently coordinated with one or more light effects generated by the one or more light sources.

5. A children's guided breathing and meditation, mindfulness, and yoga toy comprising:
a sitting yogi having a first surface, a second surface, a cavity, and a visor opening, wherein the sitting yogi comprises:
a first sound push button on the first surface of the sitting yogi;
an on/off push button on the first surface of the sitting yogi;
a second sound push button on the first surface of the sitting yogi;
one or more light sources each independently positioned within the cavity; and
a translucent visor covering the visor opening;
a sound device positioned within the cavity of the sitting yogi;
wherein the sound device comprises:
a flash memory integrated circuit;
a microcontroller;
an amplifier; and
a power source,
wherein the microcontroller is in electrical communication with the first sound push button, the on/off push button, the second sound push button, the amplifier, and a power source,
wherein the microcontroller is programmed to have one or more nature sounds recordings, one or more guided breathing and meditation recordings, one or more guided mindfulness recordings, and one or more guided yoga recordings provided using a child's voice,
wherein the amplifier is in electrical communication with a speaker positioned within the cavity of the sitting yogi and the flash memory integrated circuit; and
wherein the one or more light sources are in electrical communication with the microcontroller.

6. The children's guided breathing and meditation, mindfulness, and yoga toy of claim 5, wherein the one or more light sources each independently comprise one or more light emitting diodes (LEDs).

7. The children's guided breathing and meditation, mindfulness, and yoga toy of claim 5, wherein the one or more nature sounds recordings, the one or more guided breathing and meditation recordings, the one or more guided mindfulness recordings, and the one or more guided yoga recordings are each independently coordinated with light generated by the one or more light sources.

8. The children's guided breathing and meditation, mindfulness, and yoga toy of claim 5, wherein the first sound push button is used to activate the sound device to broadcast one or more nature sounds recordings through the speaker and to activate the one or more light sources to emit one or more colors.

9. The children's guided breathing and meditation, mindfulness, and yoga toy of claim 5, wherein the one or more nature sounds recordings are each independently coordinated with one or more light effects generated by the one or more light sources.

10. The children's guided breathing and meditation, mindfulness, and yoga toy of claim 5, wherein one or more light effects cause the one or more light sources to fade in and to fade out.

11. The children's guided breathing and meditation, mindfulness, and yoga toy of claim 5, wherein the on/off push button is used to deactivate the sound device and to deactivate the one or more light sources.

12. The children's guided breathing and meditation, mindfulness, and yoga toy of claim 5, wherein the second sound push button is used to activate the sound device to broadcast the one or more guided breathing and meditation recordings, the one or more guided mindfulness recordings, and the one or more guided yoga recordings through the speaker and to activate the one or more light sources to emit one or more colors.

13. The children's guided breathing and meditation, mindfulness, and yoga toy of claim 5, wherein the one or more guided breathing and meditation recordings, the one or more guided mindfulness recordings, and the one or more guided yoga recordings are each independently coordinated with one or more light effects generated by the one or more light sources.

14. The children's guided breathing and meditation, mindfulness, and yoga toy of claim 13, wherein one or more light effects cause the one or more light sources to fade in and to fade out.

15. The children's guided breathing and meditation, mindfulness, and yoga toy of claim 5, wherein the sitting yogi comprises one or more metals, one or more plastics, one or more wooden materials, or a combination thereof.

16. The children's guided breathing and meditation, mindfulness, and yoga toy of claim 15, wherein the sitting yogi comprises one or more plastic materials.

17. The children's guided breathing and meditation, mindfulness, and yoga toy of claim 15, wherein the sitting yogi comprises one or more rigid plastic materials.

18. The children's guided breathing and meditation, mindfulness, and yoga toy of claim 5, wherein the one or more nature sounds recordings each independently comprise one or more ocean sounds, one or more forest sounds, one or more space sounds, one or more animal sounds, one or more wind sounds, or a combination thereof.

19. A children's guided breathing and meditation, mindfulness, and yoga toy comprising:
a sitting yogi having a first surface, a second surface, a cavity, and a visor opening, wherein the sitting yogi comprises:
a first sound push button on the first surface of the sitting yogi;
an on/off push button on the first surface of the sitting yogi;
a second sound push button on the first surface of the sitting yogi;
one or more light emitting diodes (LEDs) each independently positioned within the cavity; and
a translucent visor covering the visor opening;
a sound device positioned within the cavity of the sitting yogi;
wherein the sound device comprises:
a flash memory integrated circuit;
a microcontroller;
an amplifier; and
a battery,
wherein the microcontroller is in electrical communication with the first sound push button, the on/off push button, the second sound push button, the amplifier, and a battery,
wherein the microcontroller is programmed to have one or more nature sounds recordings, one or more guided breathing and meditation recordings, one or more guided mindfulness recordings, and one or more guided yoga recordings provided using a child's voice,
wherein the amplifier is in electrical communication with a speaker positioned within the cavity of the sitting yogi and the flash memory integrated circuit;
wherein the one or more light emitting diodes (LEDs) are in electrical communication with the microcontroller, and
wherein the one or more nature sounds recordings, the one or more guided breathing and meditation recordings, the one or more guided mindfulness recordings, and the one or more guided yoga recordings are each independently coordinated with light generated by the one or more light emitting diodes (LEDs).

20. The children's guided breathing and meditation, mindfulness, and yoga toy of claim 19, further comprising one or more universal serial bus (USB) ports.
